(19) United States
(12) Patent Application Publication
(10) Pub. No.: US 2013/0064862 A1
(43) Pub. Date: Mar. 14, 2013

(54) SYSTEMS AND METHODS FOR SHELL ENCAPSULATION

(71) Applicants: David A. Weitz, Bolton, MA (US); Ho Cheung Shum, Hong Kong (HK); Yuanjin Zhao, Nanjing (CN); Bingjie Sun, Shanghai (CN); Christian Holtze, Frankfurt (DE)

(72) Inventors: David A. Weitz, Bolton, MA (US); Ho Cheung Shum, Hong Kong (HK); Yuanjin Zhao, Nanjing (CN); Bingjie Sun, Shanghai (CN); Christian Holtze, Frankfurt (DE)

(73) Assignees: BASF SE, Ludwigshafen (DE); President and Fellows of Harvard College, Cambridge, MA (US)

(21) Appl. No.: 13/586,628

(22) Filed: Aug. 15, 2012

Related U.S. Application Data
(60) Provisional application No. 61/529,126, filed on Aug. 30, 2011.

(51) Int. Cl. A61K 9/00; C09K 3/00

(52) U.S. Cl. 424/400; 252/183.11

(57) ABSTRACT

Certain aspects of the invention are generally directed to particles comprising a shell and an interior at least partially contained by the shell. In some embodiments, the particles may be treated to enhance the containment of the interior, for example to reduce transport of an agent into or out of the interior. Such particles may exhibit increased ability to encapsulate agents and/or increased storage life (e.g., due to reduced leakage). For instance, in certain embodiments, any defects, such as cracks, pores, etc. within the shell may be sealed or otherwise treated to reduce transport therethrough. In some embodiments, for instance, a first reactant in the interior of a particle may come into contact with a second reactant outside of the particle to form a solid, or other suitable product. The shell may also be treated to cause release of an agent contained within the interior, in certain aspects.

### RELATED APPLICATIONS

This application claims the benefit of U.S. Provisional Patent Application Serial No. 61/529,126, filed Aug. 30, 2011, entitled “Systems and Methods for Shell Encapsulation,” by Weitz, et al., incorporated herein by reference.

### GOVERNMENT FUNDING

Research leading to various aspects of the present invention were sponsored, at least in part, by the NSF, Grant No. DMR-1006546 and MRSEC, Grant No. DMR-0820484. The U.S. Government has certain rights in the invention.

### FIELD OF INVENTION

The present invention generally relates to particles, including particles for drug delivery and other applications.

### BACKGROUND

Microparticles such as microcapsules have great potential for applications involving encapsulation, delivery, and release of agents in fields such as agriculture, health care, cosmetics and detergents, construction chemicals, and food and beverages. A variety of physical and chemical methods, including spray-drying, coextrusion, interfacial polymerization, and complex coacervation, have been used for high-throughput preparation of microparticles. For example, using various microfluidic technologies, a variety of agents have been encapsulated into various double or other multiple emulsions, which are then solidified to form solid microparticles or other types of particles, for instance, by interfacial polycondensation, freezing, or polymerization of one or more phases of the multiple emulsion, for example, a middle phase encapsulating an inner phase. However, leakage of the agent is often observed. Such leakage can decrease encapsulation efficiency, or shorten the useful life of the agent or the particles. Accordingly, improvements in particle technologies are still needed.

### SUMMARY OF THE INVENTION

The present invention generally relates to particles, including particles for drug delivery and other applications. The subject matter of the present invention involves, in some cases, interrelated products, alternative solutions to a particular problem, and/or a plurality of different uses of one or more systems and/or articles.

In one aspect, the present invention is generally directed to an article. In accordance with one set of embodiments, the article comprises a fluid containing a microparticle comprising a shell formed from a shell material and an interior at least partially contained by the shell. The interior may contain a first reactant and the fluid containing a second reactant. In some cases, one or both of the first reactant and the second reactant is able to move towards the other. In certain instances, the first reactant and the second reactant are able to react to form a product.

In another set of embodiments, the article includes a microparticle comprising a shell and an interior at least partially contained by the shell. The shell may be formed from a shell material and contain, within the shell material, a first reactant and a second reactant able to react with the first reactant to produce a product.

The article, according to another set of embodiments, may include a fluid containing a microparticle comprising a shell and an interior at least partially contained by the shell. The shell may be formed from a shell material and contain, within the shell material, a product. In some cases, the product is formed from a first reactant and a second reactant. The first reactant may be soluble in the interior, the second reactant may be soluble in the fluid, and/or the product may be insoluble in both the interior and the fluid.

In accordance with still another set of embodiments, the article may comprise a microparticle comprising a shell and an interior at least partially contained by the shell, the shell formed from a shell material and containing, within the shell material, CaCO₃.

In another aspect, the present invention is generally directed to a method. In one set of embodiments, the method includes acts of providing a microparticle comprising a shell and an interior at least partially contained by the shell, where the interior contains a first reactant, and exposing the microparticle to a fluid containing a second reactant. In some embodiments, one or both of the first reactant and the second reactant moves towards the other to react to form a product.

The method, according to another set of embodiments, include acts of providing a microparticle comprising a shell and an interior at least partially contained by the shell, where the interior and at least a portion of the shell contains a first reactant, and exposing the microparticle to a second reactant. In some instances, the second reactant is able to react with the first reactant to form a product.

In one aspect, the present invention is generally directed to a self-sealing microparticle. In another aspect, the present invention is generally directed to a method comprising providing a microparticle comprising a shell and an interior at least partially contained by the shell, where the interior contains an agent, and treating at least a portion of the shell to slow the release of agent from the interior of the microparticle.

In another aspect, the present invention encompasses methods of making one or more of the embodiments described herein, for example, a particle comprising a shell and an interior at least partially contained by the shell. In still another aspect, the present invention encompasses methods of using one or more of the embodiments described herein, for example, a particle comprising a shell and an interior at least partially contained by the shell.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C illustrate the formation of various particles in accordance with certain embodiments of the invention;

FIG. 2 illustrates leakage of dyes from certain particles of the invention as a function of time;

FIG. 3 illustrates leakage of dyes from particles after various times in certain embodiments of the invention;

FIGS. 4A-4F illustrate release of dyes from particles after such release is triggered;

FIG. 5 schematically illustrates the blockage of defects in particles, in accordance with certain embodiments of the invention;

FIG. 6 illustrates the leakage of dyes from certain particles of the invention as a function of storage time; and

FIGS. 7A-7B illustrate EDX spectroscopy of certain particles of the invention.

### DETAILED DESCRIPTION

The present invention generally relates to particles, including particles for drug delivery and other applications. Certain aspects of the invention are generally directed to particles comprising a shell and an interior at least partially contained by the shell. In some embodiments, the particles may be treated to enhance the containment of the interior, for example to reduce transport of an agent into or out of the interior. Such particles may exhibit increased ability to encapsulate agents and/or increased storage life (e.g., due to reduced leakage). For instance, in certain embodiments, any defects, such as cracks, pores, etc. within the shell may be sealed or otherwise treated to reduce transport therethrough, for example, with a solid. In some embodiments, for instance, a first reactant in the interior of a particle may come into contact with a second reactant outside of the particle to form a solid, or other suitable product. The shell may also be treated, e.g., at a later point in time, to cause release of an agent contained within the interior, in certain aspects. For example, the shell may be heated to cause the release of the agent from the particle, or the shell may be exposed to chemical or enzymatic degradation, or a change in osmolarity, to cause release of an agent. Still other aspects of the present invention are generally directed to methods of making or using such particles, kits or devices including such particles, or the like.

Various aspects of the present invention are generally directed to particles, such as microparticles, comprising a shell and an interior at least partially contained by the shell, where the shell can be treated to reduce defects, and/or to reduce the transport of agents into and/or out of the interior of the particle. For instance, in one set of embodiments, a particle may comprise a shell and an interior containing a first reactant, and the particle may be exposed to a fluid containing a second reactant. The first reactant and/or the second reactant may be able to move into or through the shell, e.g., through a defect, such as a crack, a pore, or a channel, and/or through the shell material itself, e.g., via diffusion. The first reactant and the second reactant may be chosen such that, when the reactants come into contact with each other, a reaction occurs to produce a product. The product may, for example, deposit in or otherwise become lodged within the defect and/or within the shell material itself. For instance, the product may precipitate within the defect to at least partially seal the defect, and/or the product may become integrated within the shell material itself, e.g., to reduce the ability of an agent entering or exiting the interior of the particle.

One non-limiting example of suitable reactants are sodium carbonate (Na₂CO₃) and calcium chloride (CaCl₂), which can react together to produce sodium chloride (NaCl) and calcium carbonate (CaCO₃). Calcium carbonate is not readily soluble in water, and may thus deposit or precipitate in solid form, e.g., within a defect and/or within the shell material itself. Other examples of reactants and products are discussed in detail below. Such particles may be used in certain embodiments, for example, to increase the ability of a particle to contain an agent therein, e.g., within an interior of the particle, by preventing the agent from being able to exit the particle, e.g., via diffusion or through a defect, etc. Such particles may exhibit, in some embodiments, increased lifetime of the agent within the particle (e.g., as measured through half-life or percentage of loss of the agent from the particle). In certain embodiments, as discussed herein, the particle is self-sealing, i.e., minor defects formed within the particle may be resealed without requiring any external control.

As mentioned, certain aspects of the invention are generally directed to particles, such as microparticles. In some embodiments, the particles as discussed herein may comprise a shell and an interior at least partially contained by the shell. However, in other embodiments, the particles as discussed below may have any other suitable configuration. As non-limiting examples, the particle may have multiple interior regions, and/or more than one shell or shell material containing an interior region. In some cases, the particle may have irregular interior cavities, and/or the particle may be relatively porous, wherein the pores define interior portions of the particles. In some embodiments, the particles may be relatively homogeneous, e.g., such that agent is substantially evenly dispersed within the particle. Thus, in the descriptions herein, it should be understood that references to an “interior” of a particle are by way of ease of convenience only, and in other embodiments, a particle may contain more than one suitable interior region, and/or the particle may contain an interior that is not necessarily spherical, but may be of any suitable shape or volume defined internally of the particle. Similarly, a “shell” of a particle may not necessarily be defined only as an exterior shell on the particle (for example, as in an eggshell), but the material that forms the shell may also extend, in certain embodiments, into interior portions of the particle, for example, defining internal walls within a particle that define more than one interior region within a particle.

The particles may include a first reactant therein, for example, in an interior region of the particle. The particle containing the first reactant may be exposed to a fluid, such as a liquid, containing a second reactant that is able to react with the first reactant to produce a product. In certain embodiments, both the first reactant and the second reactant are able to move into or through a shell of a particle, e.g., through a defect and/or through the shell material itself. The first reactant and/or the second reactant may move through the same or different mechanisms, for example, via diffusion, osmolarity differences, convection, concentration gradients, differences in temperature or pressure, or the like. For example, the first reactant and the second reactant may each move through defects in the shell of a particle (e.g., through cracks, channels, holes, voids, pores, etc. within the shell) to come into direct physical contact with each other. The defects, if present, may be present when the particle is formed, and/or subsequently introduced, e.g., via during use of the particles, and/or intentionally introduced in the particles. In some embodiments, the rate of release of the agent from the particle may be controlled, for example, by osmolarity differences or concentration differences, e.g., between the interior of the particle and the fluid surrounding the particle.

In some cases, as discussed herein, the product formed from the reaction of the reactants may at least partially or completely clog or seal such defects. For example, product may be deposited within the defect, e.g., through transport of the first reactant and the second reactant through the defect, such that the product fully blocks the defect and seals the defect from further transport through the defect, for example, of an agent. In some embodiments, however, the product may not necessarily fully block the defect, but nonetheless be able to clog or impede transport through the defect, e.g., of an agent. In certain cases, the first reactant and the second reactant may each be able to move through the shell material itself (for example, via diffusion) to come into direct physical contact with each other within the shell material. In certain embodiments, more than one pathway may be used. For instance, the first reactant and/or the second reactant may move through both defects and the shell material itself, and/or one may move through a defect while the other may move through the shell material itself.

Accordingly, certain embodiments are directed to self-sealing particles, i.e., any defect formed within the particle may be resealed without requiring any external control from a user, e.g., in response to the formation of the defect. Thus, in certain embodiments, a defect occurring in such self-sealing particles allows the first reactant and the second reactant to come into contact with each other (e.g., within the defect), which causes the formation of product within the defect and thus may cause the defect to become sealed. As a specific non-limiting example, the particle may comprise an interior containing a first reactant, where the particle is exposed to a fluid containing a second reactant. If no defects are present, the shell material of the particle substantially prevents the first reactant and the second reactant from coming into direct physical contact with each other, and thus no reaction occurs that can produce a product. However, the creation of a defect may create a transport pathway that allows the first reactant and the second reactant to come into contact with each other through the transport pathway, thereby resulting in their reaction and formation of a product, which may deposit in or otherwise become lodged within the defect, thereby causing partial or complete sealing of the defect without requiring any external control from a user, for instance, in response to the formation or detection of the defect.

In some embodiments, however, only one of the first reactant and the second reactant is able to move into or through the shell material, e.g., due to hydrophobic effects, size limitations, tortuosity limitations, trapped pockets of gas or fluid, or the like. For example, in one embodiment, only a first reactant contained within an interior of a particle is able to move into or through the shell material, and upon reaching the exterior of the particle where the second reactant is located, the first reactant and the second reactant are able to react to produce a product. The product may be present within the shell material (e.g., dissolved) or otherwise contained within the shell material (e.g., within pores or defects within the shell material). As another non-limiting example, in another embodiment, a first reactant contained within an interior of the particle may be unable to substantially exit the particle through the shell material, while a second reactant is able to move into or through the shell material to come into contact with the first reactant in the interior of the particle.

In some embodiments, most of the first reactant and the second reactant within the shell material may react to from product, i.e., such that there is relatively little unreacted first reactant and/or second reactant present within the shell material. In some embodiments, for example, the shell material may contain product having a mass that is at least the sum of the masses of the first reactant and the second reactant, and in some cases, at least 3 times, at least 5 times, at least 7 times, at least 10 times, or at least 25 times the sum of the masses of the first reactant and the second reactant. Thus, in some embodiments, the shell material may contain relatively large amounts of product but relatively little amounts of unreacted reactant. In other embodiments, however, there may be greater amounts of first reactant and/or second reactant present within the shell material, for example, due to differences in concentration of the first reactant and the second reactant between the interior of the particle and fluids surrounding the particle, reaction rates (for example, a relatively slow reaction rate between the first reactant and the second reactant), or time.

The first reactant and the second reactant may be chosen such that when the reactants come into contact with each other, a reaction occurs that produces a product. In some embodiments, the reaction is spontaneous, or the reaction may also be catalyzed by a catalyst or an enzyme in certain embodiments. The product may be any suitable product that can be formed on or in the shell material; for example, the product may be a solid, a salt (for example, one which is substantially insoluble in a fluid contacting the particle and/or an interior region of the particle). For example, the product may be one that has a solubility of less than about 10 wt %, less than about 5 wt %, less than about 3 wt %, less than about 1 wt %, less than about 0.5 wt %, less than about 0.3 wt %, or less than 0.1 wt %, e.g., in the shell material and/or in the fluid contacting the particle and/or an interior region of the particle. In some cases, the solubility of the product may be chosen to be less than the solubility of at least one agent (or in some cases, all agents) contained within the particle, and/or other materials forming the particle. The product may be organic or inorganic, depending on the reaction. Similarly, the first reactant and/or the second reactant may each independently be, for instance, a salt, an organic compound, or the like. In some cases, the first reactant and/or the second reactant may be dissolved and/or suspended, e.g., in an interior region, in shell material, and/or in a fluid surrounding the particle. The reaction between the first reactant and the second reactant may be any suitable reaction, and may not necessarily involve the formation of covalent bonds; for example, reactions may also include precipitation, polymerization, decomposition, displacement, or other suitable types of reactions.

A reactant may be present in any suitable form, e.g., in a fluid surrounding the particle, or in an interior of the particle. For example, the reactant may be dissolved, carried as a dispersion or a suspension, etc. Non-limiting examples of solvents or other fluids that may be used to contain a reactant (e.g., in a fluid surrounding the particle, or in an interior of the particle) include alcohols, such as ethanol, methanol, 1-propanol, 2-propanol, or the like. Other examples of suitable solvents or other fluids include, but are not limited to, 1,2-butanediol, ethylene glycol, propylene glycol, glycerol, and/or water (i.e., producing an aqueous solution when water is used as a solvent). The water may also be present, for example, as a salt solution. Still other examples include polar aprotic solvents such as tetrahydrofurane, acetone, dimethyl sulfoxide, N,N-dimethylformaide, or the like; acidic compounds such as formic acid or acetic acid, etc.; or ethers such as glycol dimethyl ether, diglycol dimethyl ether, glycol methyl ether, diglycol methyl ether, 1-methoxy-2-butanol, etc. The first reactant and the second reactant may be contained in separate fluids, and the fluids may have the same or different compositions; for example, a first reactant may be contained in a first fluid and a second reactant may be contained in a second fluid, and the first and second fluids may the same or different, depending on the embodiment. As used herein, a “fluid” generally refers to a substance that tends to flow and to conform to the outline of its container, i.e., a liquid, a gas, a viscoelastic fluid, etc., and is intended to include not only a pure species, but also mixtures of two or more species, each of which may be present in any form and in any concentration. For example, a fluid containing a reactant may consist essentially of water, water containing dissolved or suspended salts or other compounds, a mixture of water and ethanol, a mixture of water and ethanol containing dissolved or suspended salts or other compounds, etc.

In one set of embodiments, a fluid containing a reactant is hydrophilic. As used herein, a “hydrophilic” fluid is a fluid that is substantially miscible in water, at least at ambient temperature (25° C.) and pressure (1 atm), such that upon mixing of the hydrophilic fluid and water, no substantial phase separation is observed over a time of at least a day. (It should be noted, of course, that water is completely miscible in itself; thus, water is a hydrophilic fluid.) As used herein, two fluids are immiscible, or not miscible, with each other when one is not soluble in the other to a level of at least 10% by weight. In some embodiments, the hydrophilic fluid may be substantially miscible in water at elevated temperatures and/or pressures. For example, the hydrophilic fluid may be substantially miscible in water at temperatures of at least about 50° C., at least about 75° C., at least about 100° C., at least about 125° C., at least about 150° C., at least about 175° C., or at least about 200° C. Relatively higher temperatures (e.g., at least about 100° C.) may be achieved, for example, at elevated pressures, e.g., pressures of at least about 2 atm, at least about 3 atm, at least about 4 atm, at least about 5 atm, at least about 6 atm, at least about 8 atm, at least about 10 atm, at least about 12 atm, at least about 14 atm, at least about 16 atm, etc.

As mentioned, the first reactant and the second reactant may react to form one or more precipitants. In certain embodiments, more than one precipitant is formed, and the various precipitants may co-precipitate separately and/or together (i.e., precipitating at the same time and/or due to the same fluidic conditions). In one set of embodiments, for instance, a first reactant (e.g., contained in the interior of a particle) may mix with a second reactant (e.g., contained in a fluid surrounding the particle).

The first reactant and the second reactant may react upon mixing to form a product. In some cases, the reaction is spontaneous. The product may form as a separate phase, and/or the product may precipitate or otherwise separate from the mixture of the first fluid and the second fluid. For example, the product may be substantially insoluble in a first fluid containing the first reactant, and/or the product may be substantially insoluble in a second fluid containing the second reactant. The product may be solid in some cases.

The reaction between the first reactant and the second reactant may be any suitable chemical reaction including, for example, an ion exchange reaction. In one set of embodiments, the reaction may be a single displacement reaction (e.g., where A+BX-->AX+B, each letter representing an ion) or a double displacement reaction (e.g., where AX+BY-->AY+BX); one of these products may be substantially insoluble, e.g., in a fluid surrounding a particle, or in an interior region of the particle. As another example, two ions may combine in solution to yield a substantially insoluble product, e.g., where A+B-->AB, as discussed herein (for example, where A and/or B are ions). In some embodiments, the product can be recovered as a separated phase or precipitant. In some cases, the reactions may be ionic reactions, where the first reactant (i.e., A or AX, respectively) is present in a dissolved state in a first fluid (e.g., in an interior of the particle) and the second reactant (i.e., BY or BX, respectively) is present in a dissolved state in a second fluid (e.g., externally of the particle).

The product formed from the mixture of the first fluid and the second fluid may be, as non-limiting examples, a polymer, an inorganic compound such as an inorganic salt, or the like. In one set of embodiments, however, the product is not a polymer. In some cases, the product may be one with a relatively low molar mass (i.e., molecular weight), e.g., of less than about 1000 Da (g/mol), less than about 500 Da, less than about 300 Da, less than about 200 Da, less than about 150 Da, or less than about 100 Da. In other embodiments, however, the product may have higher molar masses, e.g., greater than about 50 Da, greater than about 100 Da, greater than about 1 kDa, greater than about 10 kDa, greater than about 100 kDa, etc., e.g., as described herein. For example, the product may comprise a polymer, an alginate, etc.

As mentioned, as one example, the first reactant may be sodium carbonate and the second reactant may be calcium chloride, or vice versa, which are able to react together to form calcium carbonate. However, other reactants may also be used in other embodiments of the invention, instead of and/or in combination with sodium carbonate and calcium chloride. For example, in some embodiments, the first reactant may include any species containing carbonate ions and the second reactant may include any species containing calcium ions (or vice versa). The carbonate ions and the calcium ions may combine to form CaCO₃, which under some conditions will precipitate. The carbonate ions may be present in any suitable form. For instance, carbonate salts such as Na₂CO₃, K₂CO₃, or (NH₄)₂CO₃, NaHCO₃, KHCO₃, (NH₄)HCO₃, etc. may be used. Similarly, the calcium ions may be present in any suitable form; for example, calcium salts such as CaCl₂ (optionally in the form of a hydrate such as CaCl₂.2H₂O), Ca(NO₃)₂, or calcium acetate may be used.

It should be noted that other inorganic precipitation reactions may also be used, e.g., to produce precipitants other than CaCO₃ (or in addition to CaCO₃). For example, other suitable precipitants include, but are not limited to, lead (II) chloride (PbCl₂), lead (II) hydroxide (Pb(OH)₂), barium phosphate (Ba₃(PO₄)₂), barium sulfate (BaSO₄), silver chloride (AgCl), silver bromide (AgBr), zinc sulfide (ZnS), silver hydroxide (AgOH), or magnesium carbonate (MgCO₃), and/or combinations of these and/or other suitable precipitation reactions. These ions may be brought together from the first reactant and the second reactant such that, when reacted together, a precipitant including these is formed. In still other embodiments, any salt that is able to precipitate may be used.

As specific non-limiting examples, lead (II) chloride may precipitate upon reaction of a chloride ion source (e.g., NaCl, HCl, KCl, LiCl, MgCl₂, etc.) and a solution comprising a lead (II) compound (e.g., Pb(NO₃)₂, Pb(CH₃COO)₂, PbCO₃, etc.); lead (II) hydroxide may precipitate upon reaction of a hydroxide source (e.g., LiOH, NaOH, KOH, etc.) and a solution comprising a lead (II) compound (e.g., Pb(NO₃)₂, Pb(CH₃COO)₂, PbCO₃, etc.); barium phosphate may precipitate upon reaction of a barium ion source (e.g., Ba(OH)₂, BaS, BaCl₂,etc.) and a solution comprising a phosphate (e.g., H₃PO₄, (NH₄)₃PO₄, NaH₂PO₄, NaH₂PO₄, etc.); barium sulfate may precipitate upon reaction of a barium ion source (e.g., Ba(OH)₂, BaS, BaCl₂,etc.) and a solution comprising a sulfate (e.g., H₂SO₄, Na₂SO₄, K₂SO₄, Li₂SO₄, etc.); silver chloride may precipitate upon reaction of a silver ion source (e.g., AgNO₃) and a chloride ion source (e.g., NaCl, HCl, KCl, LiCl, MgCl₂, etc.); silver bromide may precipitate upon reaction of a silver ion source (e.g., AgNO₃) and a bromide ion source (e.g., NaBr, HBr, KBr, LiBr, MgBr₂, etc.); zinc sulfide may precipitate upon reaction of a zinc ion source (e.g., ZnSO₄) and a sulfide ion source (e.g., H₂S, Li₂S, Na₂S, K₂S, etc.); silver hydroxide may precipitate upon reaction of a silver ion source (e.g., AgNO₃) and a hydroxide source (e.g., LiOH, NaOH, KOH, etc.); or magnesium carbonate may precipitate upon reaction of a magnesium ion source (e.g., Mg(OH)₂, MgSO₄, MgCl₂, etc.) and a carbonate source (e.g., Na₂CO₃, K₂CO₃, (NH⁴)₂CO₃, NaHCO₃, KHCO₃, (NH₄)HCO₃, etc.).

It should also be noted, however, that the present invention is not limited to only inorganic precipitants (where an inorganic compound is one that does not contain any C—H covalent bonds, although in some cases, the inorganic compound may contain carbon atoms, such as CaCO₃, and/or hydrogen atoms, such as HCl, Ca(HCO₃)₂, or H₂CO₃). One non-limiting example of a precipitant involving organic compounds is calcium alginate. In one set of embodiments, for instance, a first (or second) reactant comprising a calcium source such as CaCl₂, Ca(NO₃)₂, calcium acetate, etc. may be combined with a second (or first) reactant comprising an alginate (e.g., sodium alginate) to form calcium alginate. As another example, the salt may include organic ions such as oxalates. Specific non-limiting examples include calcium oxalate or magnesium oxalate. For example, an oxalate may be precipitate upon reaction of an alkali metal oxalate with a calcium ion source (e.g., CaCl₂, Ca(NO₃)₂, calcium acetate, etc.) and/or a magnesium ion source (e.g., Mg(OH)₂, MgSO₄, MgCl₂, etc.).

As another example, the precipitant may be a polymer. Examples of suitable polymers include, but are not limited to, resinous polymers (e.g., melamine and formaldehyde), radical polymers (e.g. methyl methacrylate or hydroxyethyl methacrylate and a radical initiator), or polyurethane or polyurea reactions (e.g., two- or more functional isocyanates with two- or more functional alcohols and/or amines). Still other examples include sol-gel type reactions (e. g. triggered by the presence of water or acid or base), or the precipitation of particles such as nanoparticles suspended in solution (e.g., triggered by a change in solvent, salt or pH). Examples of sol-gel reactions include, but are not limited to, silanes, for example, a fluorosilane (i.e., a silane containing at least one fluorine atom) such as heptadecafluorosilane, or other silanes such as methyltriethoxy silane (MTES) or a silane containing one or more lipid chains, such as octadecylsilane or other CH₃(CH₂)ₙ-silanes, where n can be any suitable integer. For instance, n may be greater than 1, 5, or 10, and less than about 20, 25, or 30. The silanes may also optionally include other groups, such as alkoxide groups, for instance, octadecyltrimethoxysilane. In some cases, the silanes may contain other groups, for example, groups such as amines, which would make the sol-gel more hydrophilic. Non-limiting examples include diamine silane, triamine silane, or N-[3-(trimethoxysilyl)propyl] ethylene diamine silane. In some cases, more than one silane may be present in the sol-gel. For instance, the sol-gel may include fluorosilanes to cause the resulting sol-gel to exhibit greater hydrophobicity, and/or other silanes (or other compounds) that facilitate the production of polymers. In some cases, materials able to produce SiO₂ compounds to facilitate polymerization may be present, for example, TEOS (tetraethyl orthosilicate). Thus, for example, a first reactant may be a silane and a second reactant may be TEOS, or vice versa. It should also be understood that the sol-gel is not limited to containing only silanes, and other materials may be present in addition to, or in place of, the silanes.

In some aspects, the particle includes an interior at least partially contained by a shell. In some embodiments, the shell may contain defects such as cracks, channels, holes, voids, pores, etc., and the defects may be present when the shell is formed and/or subsequently introduced into the shell. The interior may contain a fluid such as a liquid or a gas in some embodiments. As mentioned, in some cases, the particle may contain multiple interior regions, and/or more than one shell or shell material containing an interior region. The shell of a particle may be formed from any suitable material. Examples of shell materials may be found, for example, in U.S. Patent Application Ser. No. 11/885,306, filed Aug. 29, 2007, entitled “Method and Apparatus for Forming Multiple Emulsions,” by Weitz, et al., published as U.S. Patent Application Publication No. 2009/0131543 on May 21, 2009, incorporated herein by reference in its entirety.

In some embodiments, for example, the shell material may comprise a wax or a gel. In certain cases, as discussed herein, the wax or gel may be heated to enter a fluidic phase, and/or cooled such that the wax or gel can form a solid phase, e.g., resulting in a capsule or a shell containing an interior. In some embodiments, a shell material may exhibit semisolid or quasi-solid properties, e.g., exhibiting a viscosity and/or a rigidity intermediate between that of a solid and a liquid, e.g., when the shell material comprises a wax and/or a gel. The shell material may also be amorphous or crystalline in some instances.

As an example of a shell material comprising a wax and/or a gel, in one set of embodiments, a multiple emulsion droplet may be formed by various techniques using a wax or gel under conditions in which the wax or gel is liquid (e.g., by forming the multiple emulsion droplet at a temperature greater than a melting point of the wax or gel), then the multiple emulsion droplet may be allowed to cool such that the wax or gel is able to at least partially solidify, e.g., such that at least part of the wax or gel becomes solid, thereby forming a shell material of a particle. For instance, if the wax or gel is formed as the outer phase of a multiple emulsion droplet, when the wax or gel is cooled to cause the wax or gel to at least partially solidify, a capsule or shell may be formed where the wax or gel encapsulates or containsan interior of the particle. Non-limiting examples of suitable waxes or gels include poly(N-isopropylacrylamide), glycerides such as fatty glycerides, paraffin oil, nonadecane, eicosane, agarose, or the like.

In some embodiments, a shell material may include (or be formed from) a material having a sol state and a gel state, such that the conversion of the shell material from the gel state into a sol state allows for the release of an agent from an interior of the shell of the particle, as discussed herein. In addition, a particle may be formed in certain embodiments through the conversion of a sol state into a gel state, e.g., of a droplet containing a material having a sol state and a gel state. As a non-limiting example, a multiple emulsion droplet may be formed where one of the outer layers of the droplet comprises a material in a sol state, then the sol state may be converted into a gel state using any suitable technique (e.g., cooling or chemical reaction), thereby forming a shell containing one or more interior regions (which each may independently be in any state, e.g., in a fluidic state, a gel state, a sol state, a solid state, etc.).

The conversion of a sol state into a gel state may be accomplished through any technique known to those of ordinary skill in the art, for instance, by cooling the material in a sol state, by initiating a chemical reaction, etc. As a specific non-limiting example, if agarose is used, a droplet containing agarose may be produced at a temperature above the gelling temperature of agarose, then the droplet subsequently cooled, causing the agarose to enter a gel state, which may in some instances be formed around one or more interior regions (for example, if the droplet is a multiple emulsion droplet). As another non-limiting example, if acrylamide is used, the acrylamide may be polymerized (e.g., using APS (ammonium persulfate) and tetramethylethylenediamine) to produce a shell material in the particle. p In another set of embodiments, a phase change can be initiated by a pressure change to produce a shell material containing an interior region. For example, a droplet (such as a multiple emulsion droplet) may be formed at a first pressure where a portion of the droplet is liquid or fluid. Decreasing or increasing the pressure to a second pressure may cause the portion to at least partially solidify, which may be sude to produce a shell material in the particle, e.g., containing an interior region. Non-limiting examples of such fluids include baroplastic polymers such as copolymers of polystyrene and poly(butyl acrylate) or poly(2-ethyl hexyl acrylate).

In another set of embodiments, a portion of a droplet may be solidified using a chemical reaction that causes solidification to occur, thereby forming a shell material containing an interior region. For example, two or more reactants added to a fluidic droplet may react to produce a solid product, e.g., as a shell material. As another example, a first reactant contained within a fluidic droplet may be reacted with a second reactant within a fluid surrounding the droplet to produce a solid, which may thus coat the droplet within a solid “shell” in some cases containing the interior region. Examples of such reactions include, but are not limited to, the reactions described above.

In yet another set of embodiments, a shell material may be formed by a polymerization reaction. Polymerization can be accomplished in a number of ways, including using a pre-polymer or a monomer that can be catalyzed, for example, chemically, through heat, via electromagnetic radiation (e.g., ultraviolet radiation), etc. to form the shell material of a particle. For instance, one or more monomer or oligomer precursors (e.g., dissolved and/or suspended within a fluidic droplet) may be polymerized to form a polymer as a shell material. The polymerization reaction may occur spontaneously, or be initiated in some fashion, e.g., during formation of a fluidic droplet, or after the fluidic droplet has been formed. For instance, the polymerization reaction may be initiated by adding an initiator to the fluidic droplet, by applying light or other electromagnetic energy to the fluidic droplet (e.g., to initiate a photopolymerization reaction), or the like, causing polymerization and formation of a shell material to occur. In some embodiments, redox initiation may be used. For example, during redox initiation, a reducing agent may be present in the interior of the particle, while an oxidizing agent may be used in the fluid surrounding the particle; exposure of the reducing agent to the oxidizing agent, e.g., as described herein, may be used to initiate the polymerization reaction. For example, certain monomers containing hydroxyl groups may undergo redox reactions with ceric ions or other oxidizing agents to form radicals capable of initiating a polymerization reaction. Additional non-limiting examples include peroxide initiators reacting with ascorbic acid or other suitable acids.

A non-limiting example of a solidification reaction is a polymerization reaction involving production of a nylon (e.g., a polyamide), for example, from a diacyl chloride and a diamine. Those of ordinary skill in the art will know of various suitable nylon-production techniques. For example, nylon-6,6 may be produced by reacting adipoyl chloride and 1,6-diaminohexane. For instance, a fluidic droplet, or portion thereof, may be solidified by reacting adipoyl chloride in the continuous phase with 1,6-diaminohexane within the fluidic droplet, which can react to form nylon-6,6 at the surface of the fluidic droplet, thereby forming a shell material, e.g., containing an interior.

Additionally, a polymer of a shell material can, in some embodiments, be degraded to return the polymer to an essentially fluid state, for example, to release an agent contained within the particle, as discussed herein. For example, a polymer may be degraded hydrolytically, enzymatically, photolytically, etc. In some embodiments, the polymer may exhibit a phase change from a solid or “glassy” phase to a “rubbery” phase, and in some cases, an agent may be able to pass through the polymer to a greater extent when the polymer is in a rubbery phase but not when the polymer is in a glassy phase. For example, the polymer may exhibit such a phase change upon being heated to at least its glass transition temperature, and in some embodiments, such heating may be performed as desired to cause release of an agent contained within the particle.

In one set of embodiments, the shell may have an average thickness (determined as an average over the particle) that is no more than about 1 mm, about 300 micrometers, about 100 micrometers, about 30 micrometers, about 10 micrometers, about 3 micrometers, about 1 micrometers, etc. In some cases, the shell may have an average thickness defined relative to the average diameter of the particle. For instance, the average thickness of the shell may be less than about 30%, less than about 25%, less than about 20%, less than about 15%, less than about 10%, less than about 5%, less than about 3%, less than about 2%, or less than about 1% of the average diameter of the particle.

The agent may be any suitable agent able to be contained within an interior of the particle, e.g., contained by a shell. For instance, a precise quantity of a drug, pharmaceutical, or other agent can be contained within a particle, e.g., in the interior of a shell, or in some instances, the agent may be a cell that is contained within a particle. Other agents that can be contained within a particle include, for example, biochemical species such as nucleic acids such as siRNA, RNAi and DNA, proteins, peptides, or enzymes, or the like. Additional agents that can be contained within a particle of the invention include, but are not limited to, nanoparticles, quantum dots, fragrances, proteins, indicators, dyes, fluorescent species, chemicals, amphiphilic compounds, detergents, drugs, or the like. Further examples of agents that can be contained within a particle of the invention include, but are not limited to, pesticides, such as herbicides, fungicides, insecticides, growth regulators, and microbicides. A particle can also serve as a reaction vessel in certain cases, such as for controlling chemical reactions, or for in vitro transcription and translation, e.g., for directed evolution technology. Non-limiting examples of fields in which particles may prove useful include food, beverage, health and beauty aids, paints and coatings, household products (e.g., detergent), and drugs and drug delivery.

Thus, as mentioned, in some embodiments, a particle containing an agent may be treated to slow the leakage of the agent from the particle, e.g., from an interior region of the particle. As specific examples, in certain embodiments, the half-life of leakage of the agent from the particle may be increased by at least about 1.5 times, at least about 2 times, at least about 3 times, at least about 5 times, at least about 10 times, at least about 20 times, at least about 30 times, at least about 50 times, at least about 100 times, etc. relative to the half-life of leakage of the agent from the particle in the absence of any treatment to the particle to slow leakage of the agent from the particle. The particle (or a portion of the particle) may be treated to slow leakage of an agent from an interior region of the particle using any suitable technique. For example, a particle may be treated by sealing any defects, such as cracks, pores, etc., for example, with a solid or other product as discussed herein, and/or by depositing a solid or other product within the shell material itself, e.g., thereby decreasing its porosity, increasing its density, or otherwise decreasing the ability of the agent to move through the shell material itself (e.g., via diffusion). Other techniques may be used to also slow leakage of the agent from the interior region of the particle, e.g., in addition to and/or instead of these techniques, in other embodiments of the invention. For example a particle may be treated by heating the particle (e.g., to seal the defects), or adding a coating to the outside of the particle.

In one aspect, an agent contained within a particle (e.g., in an interior region) may be released or “triggered” as desired. Thus, for example, the agent may be released upon exposure to a suitable external stimulus, or at a suitable time or location. Examples of stimuli include, but are not limited to, heating of the particle (e.g., to a temperature greater than a melting temperature or a glass transition temperature), or exposing the particle to a chemical that reacts with the shell, e.g., to cause hydrolytic, chemical, enzymatic, or photolytic degradation. As another example, the particle may be exposed to a change in osmolarity to cause release of agent. In some cases, dilution of the fluid surrounding the particle (e.g., with pure water, a dilute salt solution, etc.) may be enough to cause a change in osmolarity sufficient to cause the release of the agent from the particle. In other embodiments, the osmolarity may be increased, e.g., by exposing the particles to a fluid containing higher salt concentrations and/or by drying the fluid containing the particles to evaporate fluid and increasing the concentration of salts, etc.

within the fluid, in order to cause release of agent from the particle.

In some cases, for example, a particle containing an agent may not release the agent (or there may be some release of the agent, e.g., via leakage), but upon a suitable external stimulus, e.g., a change in temperature, the particle may begin releasing the agent (or the particle may release the agent at a significantly greater rate). For example, a change in temperature may cause a shell material within the particle containing an agent to at least partially liquefy or enter a gel state, which may allow (or increase) release of the agent from the particle. Other examples of suitable stimuli for triggering release of an agent from the particle are described herein, e.g., concentration, osmolarity, etc. In some embodiments, the release of agent may be controlled, for example, to be faster or slower, by controlling the external stimulus to which the particles are exposed to. For example, larger changes in concentration, temperature, etc. as discussed herein may cause more rapid release of agent, while smaller changes in concentration, temperature, etc. may cause slower release of the agent.

In certain aspects, a plurality of droplets or particles may be produced as discussed herein, and in some embodiments, droplets or particles formed therefrom may be of substantially the same shape and/or size (i.e., “monodisperse”), or of different shapes and/or sizes, depending on the particular application.

One set of embodiments is generally directed to a monodisperse distribution of droplets or particles. The shape and/or size of the fluidic droplets, or particles produced therefrom can be determined, for example, by measuring the average diameter or other characteristic dimension of the droplets or particles. As discussed herein, the droplets may be at least partially solidified to form solid particles, for example, forming a shell surrounding an interior of the particle. The “average diameter” or “average dimension” of a plurality or series of droplets or particles is the arithmetic average of the average diameters of each of the droplets or particles. Those of ordinary skill in the art will be able to determine the average diameter (or other characteristic dimension) of a plurality or series of droplets or particles, for example, using laser light scattering, microscopic examination, or other known techniques. The average diameter of a single droplet or particle, in a non-spherical particle, is the diameter of a perfect sphere having the same volume as the droplet or particle. The average diameter of a droplet or particle (and/or of a plurality or series of droplets or particles) may be, for example, less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases. The average diameter may also be at least about 1 micrometer, at least about 2 micrometers, at least about 3 micrometers, at least about 5 micrometers, at least about 10 micrometers, at least about 15 micrometers, or at least about 20 micrometers in certain cases.

In some cases, the largest dimension of the droplet or particle may be selected to be no more than about 50 micrometers, no more than about 30 micrometers, no more than about 10 micrometers, no more than about 5 micrometers, no more than about 3 micrometers, no more than about 1 micrometer, no more than about 500 nm, no more than about 300 nm, no more than about 100 nm, no more than about 50 nm, no more than about 30 nm, or no more than about 10 nm. In one embodiment, the particle has a largest dimension of at least about 5 nm, at least about 10 nm, at least about 30 nm, at least about 100 nm, at least about 300 nm, at least about 1000 nm, etc. The sizes or diameters of the particles may be determined using any suitable technique, for example, visual or electron microscopy, laser light scattering, BET, or the like. In another set of embodiments, the plurality of droplets or particles has an overall average diameter and a distribution of diameters such that no more than about 5%, no more than about 2%, or no more than about 1% of the particles have a diameter less than about 90% (or less than about 95%, or less than about 99%) and/or greater than about 110% (or greater than about 105%, or greater than about 101%) of the overall average diameter of the plurality of particles. In some embodiments, the plurality of particles has an overall average diameter and a distribution of diameters such that the coefficient of variation of the cross-sectional diameters of the particles is less than about 10%, less than about 5%, less than about 2%, between about 1% and about 10%, between about 1% and about 5%, or between about 1% and about 2%. The coefficient of variation can be determined by those of ordinary skill in the art, and may be defined as the standard deviation divided by the mean.

The rate of production of droplets (or particles) may be, in some embodiments, between approximately 100 Hz and 10,000 Hz, or between approximately 100 Hz and 5,000 Hz in certain embodiments. In some cases, the rate of droplet production may be at least about 200 Hz, at least about 300 Hz, at least about 500 Hz, at least about 750 Hz, at least about 1,000 Hz, at least about 2,000 Hz, at least about 3,000 Hz, at least about 4,000 Hz, at least about 5,000 Hz, at least 10,000 Hz, etc. In addition, production of large quantities of droplets or particles can be facilitated by the parallel use of multiple devices in some instances. In some cases, relatively large numbers of devices may be used in parallel, for example at least about 10 devices, at least about 30 devices, at least about 50 devices, at least about 75 devices, at least about 100 devices, at least about 200 devices, at least about 300 devices, at least about 500 devices, at least about 750 devices, or at least about 1,000 devices or more may be operated in parallel. The devices may comprise different channels, orifices, microfluidics, etc. In some cases, an array of such devices may be formed by stacking the devices horizontally and/or vertically. The devices may be commonly controlled, or separately controlled, and can be provided with common or separate sources of fluids, depending on the application.

As mentioned, the term “fluid” generally refers to a substance that tends to flow and to conform to the outline of its container, i.e., a liquid, a gas, a viscoelastic fluid, etc. However, as discussed elsewhere herein, one of ordinary skill in the art would recognize that a fluid may undergo a phase change (e.g., from liquid to solid). Typically, fluids are materials that are unable to withstand a static shear stress, and when a shear stress is applied, the fluid experiences a continuing and permanent distortion. The fluid may have any suitable viscosity that permits flow. If two or more fluids are present, each fluid may be independently selected among essentially any fluids (liquids, gases, and the like) by those of ordinary skill in the art, by considering the relationship between the fluids. In some cases, the droplets or particles may be contained within a carrier fluid, e.g., a liquid.

In one aspect of the present invention, multiple emulsions are formed by flowing fluids through one or more conduits. The system may be a microfluidic system. “Microfluidic,” as used herein, refers to a device, apparatus, or system including at least one fluid channel having a cross-sectional dimension of less than about 1 millimeter (mm), and in some cases, a ratio of length to largest cross-sectional dimension of at least 3:1. One or more conduits of the system may be a capillary tube. In some cases, multiple conduits are provided, and in some embodiments, at least some are nested, as described herein. The conduits may be in the microfluidic size range and may have, for example, average inner diameters, or portions having an inner diameter, of less than about 1 millimeter, less than about 300 micrometers, less than about 100 micrometers, less than about 30 micrometers, less than about 10 micrometers, less than about 3 micrometers, or less than about 1 micrometer, thereby providing droplets having comparable average diameters. One or more of the conduits may (but not necessarily), in cross-section, have a height that is substantially the same as a width at the same point. A conduit may include an opening that may be smaller, larger, or the same size as the average diameter of the conduit. For example, conduit openings may have diameters of less than about 1 mm, less than about 500 micrometers, less than about 300 micrometers, less than about 200 micrometers, less than about 100 micrometers, less than about 50 micrometers, less than about 30 micrometers, less than about 20 micrometers, less than about 10 micrometers, less than about 3 micrometers, etc. In cross-section, the conduits may be rectangular or substantially non-rectangular, such as circular or elliptical. The conduits of the present invention may also be disposed in or nested in another conduit, and multiple nestings are possible in some cases. In some embodiments, one conduit may be concentrically retained in another conduit and the two conduits are considered to be concentric. However, one concentric conduit may be positioned off-center with respect to another, surrounding conduit, i.e., “concentric” does not necessarily refer to tubes that are strictly coaxial.

Non-limiting examples of systems for creating droplets, including multiple emulsion droplets may be found in International Patent Publication Number WO 2004/091763, filed Apr. 9, 2004, entitled “Formation and Control of Fluidic Species,” by Link et al.; International Patent Publication Number WO 2004/002627, filed Jun. 3, 2003, entitled “Method and Apparatus for Fluid Dispersion,” by Stone et al.; International Patent Publication Number WO 2006/096571, filed Mar. 3, 2006, entitled “Method and Apparatus for Forming Multiple Emulsions,” by Weitz et al.; International

Patent Publication Number WO 2005/021151, filed Aug. 27, 2004, entitled “Electronic Control of Fluidic Species,” by Link et al.; International Patent Publication Number WO 2010/104604, filed Mar. 12, 2010, entitled “Method for the Controlled Creation of Emulsions, Including Multiple Emulsions,” by Weitz et al.; International Patent Publication Number WO 2011/028760, filed Sep. 1, 2010, entitled “Multiple Emulsions Created Using Junctions,” by Weitz et al.; and International Patent Publication Number WO 2011/028764, filed Sep. 1, 2010, entitled “Multiple Emulsions Created Using Jetting and Other Techniques,” by Weitz et al; each of which is incorporated herein by reference in its entirety.

A variety of materials and methods, according to certain aspects of the invention, may be used to form systems (such as those described above) configured to produce the multiple emulsions and/or particles described herein. In some cases, the various materials selected lend themselves to various methods. For example, various components of the invention are configured from solid materials, in which the conduits are configured via micromachining, film deposition processes such as spin coating and chemical vapor deposition, laser fabrication, photolithographic techniques, etching methods including wet chemical or plasma processes, injection molding, hot embossing, and the like. See, for example, Scientific American, 248:44-55, 1983 (Angell, et al). In one embodiment, at least a portion of the fluidic system is formed of silicon by etching features in a silicon chip. Technologies for precise and efficient fabrication of various fluidic systems and devices of the invention from silicon are known. In another embodiment, various components of the systems and devices of the invention are configured of a polymer, for example, an elastomeric polymer such as polydimethylsiloxane (“PDMS”), polytetrafluoroethylene (“PTFE” or Teflon®), or the like.

Different components can be fabricated of different materials. For example, a base portion including a bottom wall and side walls can be fabricated from an opaque material such as silicon or PDMS, and a top portion can be fabricated from a transparent or at least partially transparent material, such as glass or a transparent polymer, for observation and/or control of the fluidic process. Components can be coated so as to expose a desired chemical functionality to fluids that contact interior conduit walls, where the base supporting material does not have a precise, desired functionality. For example, components can be fabricated as illustrated, with interior conduit walls coated with another material. Material used to fabricate various components of the systems and devices of the invention, e.g., materials used to coat interior walls of fluid conduits, may desirably be selected from among those materials that will not adversely affect or be affected by fluid flowing through the fluidic system, e.g., material(s) that is chemically inert in the presence of fluids to be used within the device. A non-limiting example of such a coating is disclosed below; additional examples are disclosed in Int. Pat. Apl. Ser. No. PCT/US2009/000850, filed Feb. 11, 2009, entitled “Surfaces, Including Microfluidic Channels, With Controlled Wetting Properties,” by Weitz, et al., published as WO 2009/120254 on Oct. 1, 2009, incorporated herein by reference.

In some embodiments, various components of the invention are fabricated from polymeric and/or flexible and/or elastomeric materials, and can be conveniently formed of a hardenable fluid, facilitating fabrication via molding (e.g. replica molding, injection molding, cast molding, etc.). The hardenable fluid may be essentially any fluid that can be induced to solidify, or that spontaneously solidifies, into a solid capable of containing and/or transporting fluids contemplated for use in and with the fluidic network. In some embodiments, the hardenable fluid comprises a polymeric liquid or a liquid polymeric precursor (i.e. a “prepolymer”). Suitable polymeric liquids include, for example, thermoplastic polymers, thermoset polymers, or mixture of such polymers heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers are utilized in some embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of the microfluidic structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric, and thus may be useful for forming very small features with relatively high aspect ratios, necessary in certain embodiments of the invention. Flexible (e.g., elastomeric) molds or masters can be advantageous in this regard.

An advantage of forming structures such as microfluidic structures of the invention from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre- oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.), incorporated herein by reference.

In some embodiments, certain microfluidic structures of the invention (or interior, fluid-contacting surfaces) may be formed from certain oxidized silicone polymers. Such surfaces may be more hydrophilic than the surface of an elastomeric polymer. Such hydrophilic conduit surfaces can thus be more easily filled and wetted with aqueous solutions. Thus, certain devices of the invention can be made with surfaces that are more hydrophilic than unoxidized elastomeric polymers.

In some embodiments, it may be desirable to make a channel surface hydrophobic. One non-limiting method for making a channel surface hydrophobic comprises contacting the channel surface with an agent that confers hydrophobicity to the channel surface. For example, in some embodiments, a channel surface may be contacted (e.g., flushed) with Aquapel (a commercial auto glass treatment) (PPG Industries, Pittsburgh, Pa.). In some embodiments, a channel surface contacted with an agent that confers hydrophobicity may be subsequently purged with air. In some embodiments, the channel may be heated (e.g., baked) to evaporate solvent that contains the agent that confers hydrophobicity.

Thus, in one aspect of the invention, a surface of a microfluidic channel may be modified to facilitate the production of emulsions such as multiple emulsions. In some cases, the surface may be modified by coating a sol-gel onto at least a portion of a microfluidic channel. As is known to those of ordinary skill in the art, a sol-gel is a material that can be in a sol or a gel state, and typically includes polymers. The gel state typically contains a polymeric network containing a liquid phase, and can be produced from the sol state by removing solvent from the sol, e.g., via drying or heating techniques. In some cases, as discussed below, the sol may be pretreated before being used, for instance, by causing some polymerization to occur within the sol.

As an example, the sol-gel coating may be made more hydrophobic by incorporating a hydrophobic polymer in the sol-gel. For instance, the sol-gel may contain one or more silanes, for example, a fluorosilane (i.e., a silane containing at least one fluorine atom) such as heptadecafluorosilane, or other silanes such as methyltriethoxy silane (MTES) or a silane containing one or more lipid chains, such as octadecylsilane or other CH₃(CH₂)ₙ-silanes, where n can be any suitable integer. For instance, n may be greater than 1, 5, or 10, and less than about 20, 25, or 30. The silanes may also optionally include other groups, such as alkoxide groups, for instance, octadecyltrimethoxysilane. In general, most silanes can be used in the sol-gel, with the particular silane being chosen on the basis of desired properties such as hydrophobicity. Other silanes (e.g., having shorter or longer chain lengths) may also be chosen in other embodiments of the invention, depending on factors such as the relative hydrophobicity or hydrophilicity desired. In some cases, the silanes may contain other groups, for example, groups such as amines, which would make the sol-gel more hydrophilic. Non-limiting examples include diamine silane, triamine silane, or N-[3-(trimethoxysilyl)propyl] ethylene diamine silane. The silanes may be reacted to form oligomers or polymers within the sol-gel, and the degree of polymerization (e.g., the lengths of the oligomers or polymers) may be controlled by controlling the reaction conditions, for example by controlling the temperature, amount of acid present, or the like. In some cases, more than one silane may be present in the sol-gel. For instance, the sol-gel may include fluorosilanes to cause the resulting sol-gel to exhibit greater hydrophobicity, and/or other silanes (or other compounds) that facilitate the production of polymers. In some cases, materials able to produce SiO₂ compounds to facilitate polymerization may be present, for example, TEOS (tetraethyl orthosilicate).

It should be understood that the sol-gel is not limited to containing only silanes, and other materials may be present in addition to, or in place of, the silanes. For instance, the coating may include one or more metal oxides, such as SiO₂, vanadia (V₂O₅), titania (TiO₂), and/or alumina (Al₂O₃).

In some instances, the microfluidic channel is constructed from a material suitable to receive the sol-gel, for example, glass, metal oxides, or polymers such as polydimethylsiloxane (PDMS) and other siloxane polymers. For example, in some cases, the microfluidic channel may be one in which contains silicon atoms, and in certain instances, the microfluidic channel may be chosen such that it contains silanol (Si—OH) groups, or can be modified to have silanol groups. For instance, the microfluidic channel may be exposed to an oxygen plasma, an oxidant, or a strong acid cause the formation of silanol groups on the microfluidic channel.

The sol-gel may be present as a coating on the microfluidic channel, and the coating may have any suitable thickness. For instance, the coating may have a thickness of no more than about 100 micrometers, no more than about 30 micrometers, no more than about 10 micrometers, no more than about 3 micrometers, or no more than about 1 micrometer. Thicker coatings may be desirable in some cases, for instance, in applications in which higher chemical resistance is desired. However, thinner coatings may be desirable in other applications, for instance, within relatively small microfluidic channels.

In one set of embodiments, the hydrophobicity of the sol-gel coating can be controlled, for instance, such that a first portion of the sol-gel coating is relatively hydrophobic, and a second portion of the sol-gel coating is relatively hydrophobic. The hydrophobicity of the coating can be determined using techniques known to those of ordinary skill in the art, for example, using contact angle measurements such as those discussed below. For instance, in some cases, a first portion of a microfluidic channel may have a hydrophobicity that favors an organic solvent to water, while a second portion may have a hydrophobicity that favors water to the organic solvent.

The hydrophobicity of the sol-gel coating can be modified, for instance, by exposing at least a portion of the sol-gel coating to a polymerization reaction to react a polymer to the sol-gel coating. The polymer reacted to the sol-gel coating may be any suitable polymer, and may be chosen to have certain hydrophobicity properties. For instance, the polymer may be chosen to be more hydrophobic or more hydrophilic than the microfluidic channel and/or the sol-gel coating. As an example, a hydrophilic polymer that could be used is poly(acrylic acid).

The polymer may be added to the sol-gel coating by supplying the polymer in monomeric (or oligomeric) form to the sol-gel coating (e.g., in solution), and causing a polymerization reaction to occur between the polymer and the sol-gel. For instance, free radical polymerization may be used to cause bonding of the polymer to the sol-gel coating. In some embodiments, a reaction such as free radical polymerization may be initiated by exposing the reactants to heat and/or light, such as ultraviolet (UV) light, optionally in the presence of a photoinitiator able to produce free radicals (e.g., via molecular cleavage) upon exposure to light. Those of ordinary skill in the art will be aware of many such photoinitiators, many of which are commercially available, such as Irgacur 2959 (Ciba Specialty Chemicals) or 2-hydroxy-4-(3-triethoxysilylpropoxy)-diphenylketone (SIH6200.0, ABCR GmbH & Co. KG).

The photoinitiator may be included with the polymer added to the sol-gel coating, or in some cases, the photoinitiator may be present within the sol-gel coating. For instance, a photoinitiator may be contained within the sol-gel coating, and activated upon exposure to light. The photoinitiator may also be conjugated or bonded to a component of the sol-gel coating, for example, to a silane. As an example, a photoinitiator such as Irgacur 2959 may be conjugated to a silane-isocyanate via a urethane bond, where a primary alcohol on the photoinitiator may participate in nucleophilic addition with the isocyanate group, which may produce a urethane bond.

It should be noted that only a portion of the sol-gel coating may be reacted with a polymer, in some embodiments of the invention. For instance, the monomer and/or the photoinitiator may be exposed to only a portion of the microfluidic channel, or the polymerization reaction may be initiated in only a portion of the microfluidic channel. As a particular example, a portion of the microfluidic channel may be exposed to light, while other portions are prevented from being exposed to light, for instance, by the use of masks or filters. Accordingly, different portions of the microfluidic channel may exhibit different hydrophobicities, as polymerization does not occur everywhere on the microfluidic channel. As another example, the microfluidic channel may be exposed to UV light by projecting a de-magnified image of an exposure pattern onto the microfluidic channel. In some cases, small resolutions (e.g., 1 micrometer, or less) may be achieved by projection techniques. In some embodiments, a bottom wall of a microfluidic device of the invention is formed of a material different from one or more side walls or a top wall, or other components. For example, in some embodiments, the interior surface of a bottom wall comprises the surface of a silicon wafer or microchip, or other substrate. Other components may, as described above, be sealed to such alternative substrates. Where it is desired to seal a component comprising a silicone polymer (e.g. PDMS) to a substrate (bottom wall) of different material, the substrate may be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques may be used, as would be apparent to those of ordinary skill in the art, including, but not limited to, the use of separate adhesives, bonding, solvent bonding, ultrasonic welding, etc.

The following documents are incorporated herein by reference in their entireties: U.S. patent application Ser. No. 11/885,306, filed Aug. 29, 2007, entitled “Method and Apparatus for Forming Multiple Emulsions,” by Weitz, et al., published as U.S. Patent Application Publication No. 2009/0131543 on May 21, 2009; U.S. Pat. No. 7,776,927, issued Aug. 17, 2010, entitled “Emulsions and Techniques for Formation,” by Chu, et al.; U.S. patent application Ser. No. 13/049,957, filed Mar. 17, 2011, entitled “Melt Emulsification,” by Shum, et al.; and U.S. Provisional Patent Application Ser. No. 61/504,990, filed Jul. 6, 2011, entitled “Systems and Methods for Forming Droplets, Including Encapsulated Droplets,” by Kim, et al. Also incorporated herein by reference in its entirety is U.S. Provisional Patent Application Ser. No. 61/529,126, filed Aug. 30, 2011, by Weitz, et al.

The following examples are intended to illustrate certain embodiments of the present invention, but do not exemplify the full scope of the invention.

### EXAMPLE 1

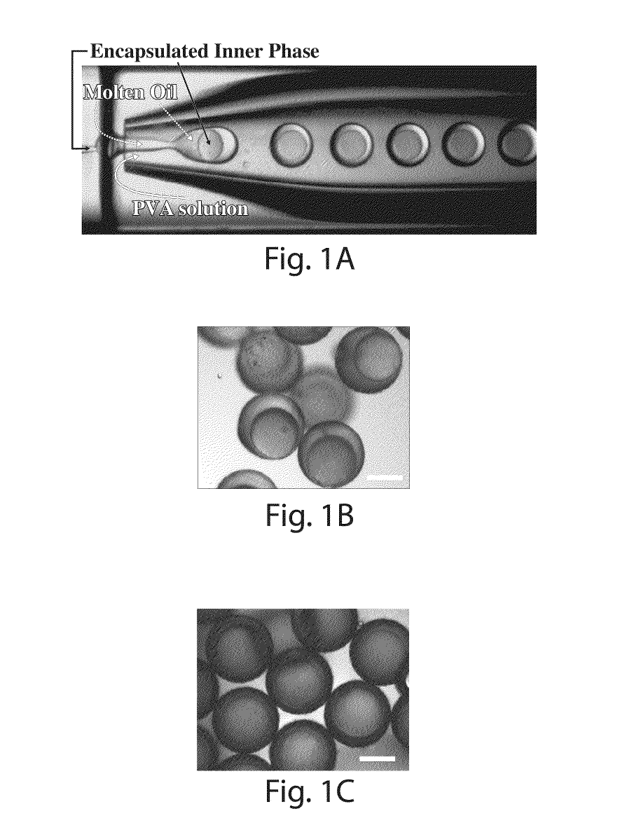

In this example, W/O/W (water/oil/water) double emulsions with a liquid middle phase were used for the fabrication of certain microparticles or “microcapsules.” The monodisperse double emulsions were generated with a glass microcapillary microfluidic device that combines a co-flow and a flow focusing geometry shown in FIG. 1A. The inner droplet aqueous fluid, containing an agent (here, a dye), was formed in the “dripping” regime using an injection tube in the co-flow geometry, while the middle oil phase that contained the inner droplet was flow-focused by an outer aqueous continuous phase from the opposite end. As a result, the jet broke up to form double emulsion droplets. Because the inner phase was in contact only with the middle oil phase, coalescence between the inner phase and the continuous phase (as both were aqueous) was prevented; thus there was no leakage of the agent to the outer continuous phase during emulsion generation. The overall size of the double emulsions, and the thickness of the shell, could be adjusted, for example, by tuning the flow rates of the fluid phases and/or the diameters of the capillaries in the device.

Solid microcapsules were obtained by cooling the middle phase of the double emulsions below its melting temperature, thereby causing the middle phase to form a solid. Because the double emulsions are thermodynamically unstable, they should be cooled quickly, and the osmotic pressure across the resulting solid phase (or shell) should be minimized. Since the innermost phase and the middle or shell phase were sometimes not density-matched, delaying solidification of the shell could sometimes result in inner droplets that were significantly off center, as shown in FIG. 1B, which is an optical micrograph of solid microcapsules obtained after collection and delayed solidification (about 20 seconds) in vials containing a 10% PVA solution at 4° C. Encapsulated agents tend to leak quickly from such microcapsules, particularly through the thinner regions of the shell, which may significantly reduce their encapsulation efficiency. Thus, in some experiments, the microcapsules were prepared by cooling the double emulsions immediately inside the collection tube, and collecting the solidified particles in an ice-water mixture or a cooled salt solution having an osmolarity generally matched to that of the innermost phase. As the microcapsules solidified almost immediately after formation, the off-centering of the inner droplets was minimized, as shown in FIG. 1C, and there was no loss of encapsulation efficiency due to leakage through the shell. This figure is an optical micrograph of solid microcapsules after collection and solidification within about 5 seconds in cold water. The scale bars denote 100 micrometers.

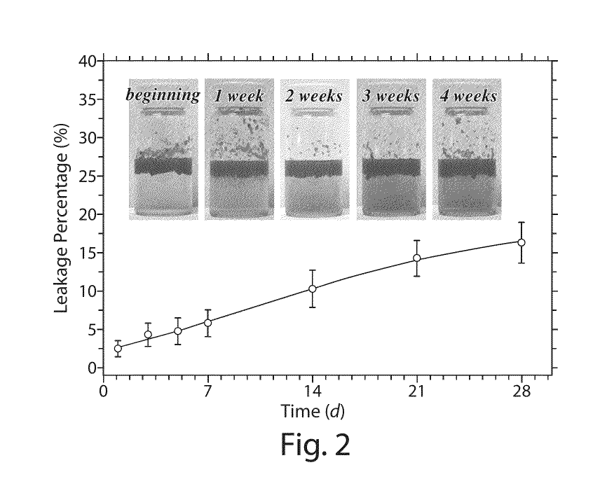

Even though the dye (representing an agent) was well encapsulated by the glycerides during emulsion generation, and the shells were not disrupted throughout the observation period, dye leakage was still observable. To visualize this leakage, a model compound, Allura Red AC food dye, was encapsulated into the fatty acid glyceride microcapsules, and leakage of the dye from the microcapsules was monitored by detecting ultraviolet/visible (“UV-vis”) absorbance of the continuous phase. An average leakage of 16.3% of the dye was observed in a time of four weeks, as shown by the gradual coloring of the continuous phase in the photographs of the capsule suspension and by the increase in UV-vis absorbance in FIG. 2, showing a plot of the Allura Red AC food dye leakage percentage from the plain microcapsules as a function of preserving times. The insets show pictures of the microcapsules in the vessels after different storage times. By increasing the thickness of the glycerides shells of the microcapsules, the leakage of the encapsulated food dye was reduced, as shown in FIG. 6, which is a plot of the leakage percentage of the dye, Allura Red AC, from the glyceride microcapsules as a function of storage time for different volume ratios of the shell and the inner phase. From top to bottom, the ratios are 1:1, 2:1, 4:1, and 6:1, respectively. However, thicker shells may reduce the amount of loading of the encapsulated agent.

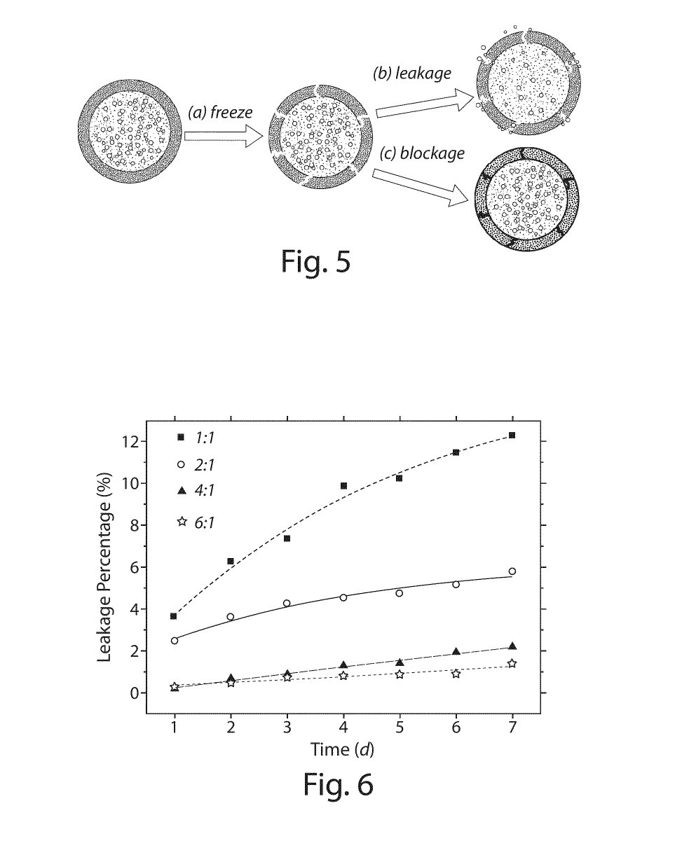

To effectively reduce this undesired leakage, two reactants of a precipitation reaction were added, one to the inner phase of the microcapsule and the second to the continuous phase outside of the microcapsule. These reactants were able to diffuse across the shell. Upon meeting in the shell, the two reactants were able to form solid precipitates, which effectively blocked the diffusion pathways as indicated in FIG. 5. In (a) in FIG. 5, small channels were present in the shell of the microcapsules, for example, created during freezing-induced solidification; (b) shows that the encapsulated agent is able to leak from pores and/or channels in the shell, while (c) shows the introduction of reactants for precipitation, which lead to blockage of the pores and channels.

To implement this concept, two common salts, sodium carbonate and calcium chloride, were dissolved into the inner phase and the continuous solution. To assess leakage, microcapsules were prepared with a 2:1 volume ratio of the glycerides and the encapsulated inner droplets. After the glycerides shells of the microcapsules solidified, the salts in the solutions of the inner droplets and the outer collection could diffuse across the shells. When the salts met, they react to form solid calcium carbonate.

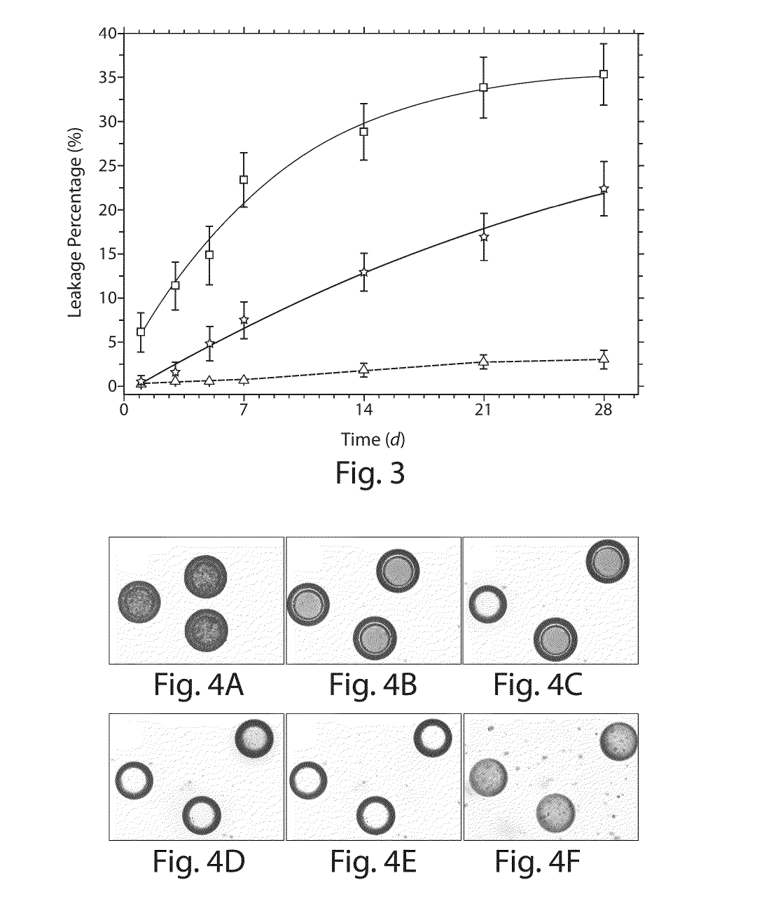

Using this approach, leakage of the dye in the microcapsules was significantly reduced, from about 16% to only about 3% in 4 weeks, as shown by the reduction in the UV-vis absorbance in FIG. 3. This figure shows plots of the food dye leakage percentage from the glyceride microcapsules as a function of storage time. Squares represent the microcapsules containing sodium carbonate solution and pure water in the inside and outside of the capsules respectively; stars represent the microcapsules with sodium carbonate solution and sodium chloride solution in the inside and outside of the capsules respectively; triangles represent the microcapsules with sodium carbonate solution and calcium chloride solution in the inside and outside of the capsules respectively. The lines are guides to the eye.

As a control, these experiments were repeated by replacing the reactants with non-reactive salts at the same concentrations. When only sodium carbonate was added to the inner phase without calcium chloride added to the continuous phase, about 35% of the dye leaked out over 4 weeks. In this case, the leakage appeared to be exacerbated by the large difference in osmolalities between the inner and the continuous phase. However, even when sodium carbonate and sodium chloride were added to the two phases with no osmotic pressure across the shell, about 22% of the dye leaked out over 4 weeks. These profiles confirmed the efficacy of the precipitation strategy in preventing the leakage of the actives from the microcapsules.

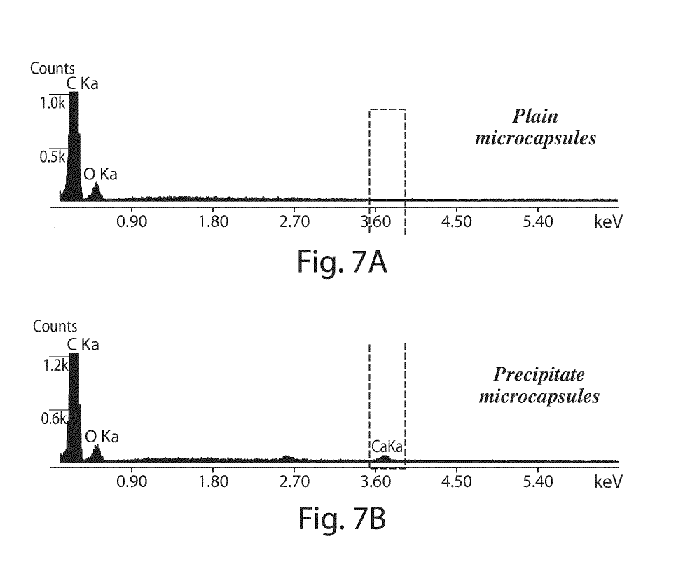

In another set of experiments, precipitates in the shell of the microcapsules were studied using elemental analysis. Calcium was detected in the solid shell of the microcapsules, as shown by the Energy-Dispersive X-ray (EDX) spectroscopy data in FIG. 7. In this figure, energy-dispersive X-ray (EDX) spectroscopy of the shells of the plain microcapsules (top) and microcapsules with precipitation reaction (bottom) are each illustrated, showing the presence of calcium in the shell. These results suggested that dye molecules leaked out of the capsules through small pores that may form upon the rapid solidification of the shell, and that may be blocked with the precipitates. Pores can indeed be observed on the surface of the capsules, as confirmed by SEM images of the microcapsules. To demonstrate the generality of this approach for different agents and shell materials, these experiments were repeated using Tartrazine and Witepsol H15 oil as alternative agents and shells, respectively. In these experiments, leakage of the contained agent was reduced significantly.

Despite being contained within the particle, the agent could be easily released from the particle upon triggering. In some experiments, after heating the microcapsular particles above their melting temperature, the shell began to melt and the microcapsules were no longer stable. The inner droplets were able to coalesce with the continuous phase, releasing the agent (dye), as shown in FIG. 4. FIGS. 4A-4E are bright-field microscope images showing the dye release from three microcapsules during heating; the whole process of release takes about 2 min The release was confirmed by the formation of solid wax spheres rather than microcapsules after refreezing, as shown in the optical micrographs in FIG. 4F. FIG. 4F is a bright field image of the re-frozen microcapsules after the triggered release of the dye, showing the solid particles that remain. Thus, these results show that, while incorporation of the precipitation reaction enhances encapsulation of agents, release of the agents can still be performed at will.

In summary, this example illustrates a novel approach for enhancing the encapsulation of agents inside microcapsules or other particles. By adding reactants for a precipitation reaction separately to the inner and the continuous phases of the microcapsules, leakage can be significantly reduced. The formation of precipitates within the shell blocks pores, slowing the leakage rate. This approach does not require any additional processing steps, yet allows fabrication of self-sealing microcapsules with highly efficient encapsulation of agents.

Following is additional information regarding these experiments.

Materials. The inner phase used in microfluidics included 1 wt % Allura Red AC or Tartrazine (Sigma-Aldrich Co.), and 1 wt % sodium carbonate. The middle oil phase was a molten Suppocire AIM oil (mixture of glycerides of saturated fatty acids from C8-C18, m.p. 33-35° C., Gatefosse) or Witepsol H15 (m.p. 33.5-35.5° C., fatty glyceride saturation C10-C18, Sasol) maintained at a constant temperature of 70° C. The outer phase was a 10 wt % poly(vinyl alcohol) (PVA; MW: 13,000-23,000 g/mol, 87-89% hydrolyzed, Sigma-Aldrich Co.). Solutions were all filtered before introduction into glass microcapillary devices. Water with a resistivity of 18.2 MΩ/cm (megohm/cm) was acquired from a Millipore Milli-Q system.

Microcapsules fabrication. The microcapsules were formed from W/O/W double emulsions. Uniform double emulsions were prepared using microcapillary devices. The round capillaries, with inner and outer diameters of 0.58 mm and 1.0 mm, were purchased from World Precision Instruments, Inc. and tapered to desired diameters with a micropipette puller (P-97, Sutter Instrument, Inc.) and a microforge (Narishige International USA, Inc.). The tapered round capillaries were fitted into square capillaries (Atlantic International Technology, Inc.) with an inner dimension of 1.0 mm for alignment. During fabrication of the double emulsion, a typical set of flow rates for the outer, middle, and inner phases was 15,000, 2,000, and 1,000 microliters/hr, respectively. All the fluids were pumped into the capillary microfluidic device using syringe pumps (Harvard PHD 2000 series). The double emulsions generated were collected into bottles which were filled with ice-water mixtures or a 1 wt % calcium chloride solution.

Characterization. The double emulsion generation process in the microfluidic device was monitored using an inverted optical microscope (DM-IRB, Leica) connected to a high-speed camera (Phantom V9, Vision Research). Bright-field images were obtained with 5×, 10×, and 20×, objectives at room temperature using an automated inverted microscope with fluorescence (Leica, DMIRBE) equipped with a digital camera (QImaging, QICAM 12-bit). The release profile of Allura Red AC and Tartrazine was monitored using a UV-vis spectrophotometer (Nanodrop, ND 1000). Scanning electron microscopic (SEM) images of dried microcapsules coated with a thin layer of platinum and palladium were taken using a Zeiss Supra 55VP field emission scanning electron microscope (FESEM, Carl Zeiss, Germany) at an acceleration voltage of 3 kV.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a fluid containing a microparticle comprising a shell formed from a shell material and an interior at least partially contained by the shell, the interior containing a first reactant and the fluid containing a second reactant, wherein one or both of the first reactant and the second reactant is able to move towards the other, and wherein the first reactant and the second reactant are able to react to form a product.

2. The article of claim 1, wherein one or both of the first reactant and the second reactant is able to move into or through the shell to form the product.

3. The article of claim 1, wherein the product is not able to substantially move out of the shell.

4. The article of claim 1, wherein the product is substantially insoluble in the fluid and the interior.

5. The article of claim 1, wherein the product is a solid.

6. The article of claim 1, wherein the product is a salt.

7. The article of claim 1, wherein the product comprises CaCO₃.

8. The article of claim 1, wherein the shell comprises a polymer.

9. The article of claim 1, wherein the interior further comprises an agent.

10. The article of claim 9, wherein the agent exhibits a half-life of leakage from the microparticle that is at least 3 times slower than a half-life of leakage of an identical microparticle that does not contain the product.

11. The article of claim 10, wherein the agent exhibits a half-life of leakage from the microparticle that is at least 10 times slower than a half-life of leakage of an identical microparticle that does not contain the product.

12. The article of claim 1, wherein the product is releaseable from the microparticle by heating the microparticle.

13. The article of claim 12, wherein the product is releaseable from the microparticle by heating the microparticle to a temperature greater than a melting temperature of the shell material.

14. The article of any one of claim 12, wherein the product is releaseable from the microparticle by heating the microparticle to a temperature greater than a glass transition temperature of the shell material.

15. An article, comprising:
a microparticle comprising a shell and an interior at least partially contained by the shell, the shell formed from a shell material and containing, within the shell material, a first reactant and a second reactant able to react with the first reactant to produce a product.

16. The article of claim_15, wherein the shell material and the product are compositionally distinguishable.

17. The article of claim 15, wherein the shell contains product having a mass that is at least 3 times the mass of the first reactant and the second reactant within the shell.

18. The article of claim 15, wherein the product is not able to substantially move out of the shell.

19-25. (canceled)

26. An article, comprising:
a fluid containing a microparticle comprising a shell and an interior at least partially contained by the shell, the shell formed from a shell material and containing, within the shell material, a product, wherein the product is formed from a first reactant and a second reactant, the first reactant being soluble in the interior, the second reactant being soluble in the fluid, and the product being insoluble in both the interior and the fluid.

27. An article, comprising:
a microparticle comprising a shell and an interior at least partially contained by the shell, the shell formed from a shell material and containing, within the shell material, CaCO₃.

28-80. (canceled)
